(19) United States
(12) Patent Application Publication
(10) Pub. No.: US 2023/0405256 A1
(43) Pub. Date: Dec. 21, 2023

(54) PORTABLE VENTILATION DEVICES AND SYSTEMS

(71) Applicant: Accubreath, Inc., Sandy, UT (US)

(72) Inventors: Spencer MADSEN, Sandy, UT (US); Mike FOGARTY, Sandy, UT (US)

(21) Appl. No.: 18/029,622

(22) PCT Filed: Sep. 29, 2021
(86) PCT No.: PCT/US2021/052570

§ 371 Date: Mar. 30, 2023

Related U.S. Application Data
(60) Provisional application No. 63/085,488, filed on Sep. 30, 2020.

(51) Int. Cl. A61M 16/06; A61M 16/00; A61M 16/08

(52) U.S. Cl. CPC A61M 16/0683; A61M 16/0066; A61M 16/0003; A61M 16/0875; A61M 2016/0027; A61M 2205/8206

(57) ABSTRACT

A portable ventilation system (100) can have a ventilation mask. The ventilation mask includes a mask body (102) having an outer surface (106) and an inner surface (108) configured for engagement with a face of a subject. The mask body (102) defines an inlet opening (110) configured to receive an airflow. The ventilation system (100) further includes a blower assembly (112) positioned in fluid communication with the inlet opening (110) of the mask body (102) to direct the airflow to the inlet opening (110). A breath delivery mechanism (104a-b), that when manually operated, activates the blower assembly (112) and delivers a breath to the subject by increasing a pressure within the ventilation mask.

### RELATED APPLICATIONS

This application claims priority to U.S. Provisional Patent Application No. 63/085,488, filed Sep. 30, 2020, which is incorporated herein by reference.

### BACKGROUND

With an aging population, medical procedures that utilize conscious sedation are being performed at an ever-increasing rate. Conscious sedation uses a moderate level of sedation to help reduce anxiety, discomfort, and pain during certain procedures, such as colonoscopies and cataract surgeries, where the patient is still spontaneously breathing. However, patients under conscious sedation often spend a large portion of the procedure at an unsafe level of ventilation. For example, research indicates that an average healthy patient can suffer 7-10 apneic events during a procedure while under conscious sedation.

Another type of respiratory support that is commonly provided is a bag-valve-mask (BVM) device which is used by emergency responders to provide manual ventilation to patients incapable of spontaneous respiration. Use of a BVM is difficult to master and requires use of both hands of the clinician as well as their full attention to provide the proper level of tidal volume and breath rate as well as to keep the patient's airway open while holding the BVM in place. Studies have shown that even well-trained clinicians tend to provide more than 2-3 times as many breaths per minute as is recommended. Also, a patient may be injured when an excessive tidal volume is provided due to excessive pressure in the lungs which can lead to traumatic brain injury, hemorrhagic shock, lung injury, or other injuries. If tidal volume is too low, the patient may not receive enough oxygen and/or carbon dioxide may build up in the patient's lungs.

### SUMMARY

Technologies are described for portable ventilation devices and systems. In one example, there is provided a portable ventilation system, comprising a ventilation mask, wherein the ventilation mask includes a mask body having an outer surface and an inner surface configured for engagement with a face of a subject, wherein the mask body defines an inlet opening configured to receive an airflow. Further, a blower assembly can be positioned in fluid communication with the inlet opening of the mask body to direct the airflow to the inlet opening of the mask body. In addition, a breath delivery mechanism may be provided, that when manually operated, activates the blower assembly to deliver a breath to the subject by increasing a pressure within the ventilation mask.

In one example of the portable ventilation system, the breath delivery mechanism includes a sensor coupled to the ventilation mask, and the sensor detects squeezing pressure applied to opposite sides of the outer surface of the mask body and activates the blower assembly to deliver the breath to the subject.

In one example of the portable ventilation system, the breath delivery mechanism includes a button directly coupled to the ventilation mask, and the button activates the blower assembly to deliver the breath to the subject. In another alternative of the button is directly coupled to the mask body.

In one example of the portable ventilation system, the system further comprises a housing configured to contain the blower assembly, wherein the housing has dimensions and an attachment that allows the housing to be attached to a fastening worn by a person.

In one example of the portable ventilation system, the system further comprises: a wrist mount configured to attach to a wrist and receive a housing containing the blower assembly for removable attachment to the wrist mount; and a tube assembly that extends between the inlet opening of the mask body and the blowing assembly, wherein the tube assembly is attached at a first end to the blowing assembly and is attached at a second end to the inlet opening of the mask body.

In one example of the portable ventilation system, the blower assembly is attached to the outer surface of the mask body and the blower assembly is in direct fluid communication with the inlet opening of the mask body.

In one example of the portable ventilation system, the system further comprises a housing containing the blower assembly and the housing configured to be removably attached to the outer surface of the mask body.

In one example of the portable ventilation system, the system further comprises a tube assembly extending between the inlet opening of the mask body and the blower assembly contained in the housing, and the tube assembly is configured to detach from the inlet opening of the mask body to allow attachment of an alternative ventilation technique.

In one example of the portable ventilation system, the system further comprises a battery assembly to power the blower assembly in response to the breath delivery mechanism being operated.

In one example of the portable ventilation system, the battery assembly includes an induction coil and rectifier for inductive charging of the battery assembly.

In one example of the portable ventilation system, the blower assembly includes a handle having a mask interface to connect with the inlet opening of the mask, and shaped to allow a user to retain the ventilation mask on a patient using a single hand, while simultaneously engaging the breath delivery mechanism with the single hand.

In one example of the portable ventilation system, the handle has a frustoconical shape which extends away from a main body of the blower assembly.

In one example of the portable ventilation system, the system further comprises a jaw support assembly configured to be placed under a subject's chin to maintain jaw posture during engagement of the inner surface of the mask body to the face of the subject. For example, the jaw support assembly may comprise a strap that extends around and behind a head of a patient.

In one example of the portable ventilation system, the mask body further comprises at least one leak opening extending between the inner surface and the outer surface of the mask body to control pressure within the ventilation mask.

In one example, there is provided a ventilation mask, comprising a mask body having an outer surface and an inner surface configured for engagement with a face of a subject. A breath delivery mechanism, that when manually operated, can be configured to activate a blower assembly to deliver a breath to the subject, wherein the mask body defines an inlet opening configured to receive air from the blower assembly.

In one example of the ventilation mask, the breath delivery mechanism includes a sensor coupled to the mask body to detect pressure applied to the outer surface of the mask body and activate the blower assembly to deliver the breath to the subject.

In one example of the ventilation mask, the breath delivery mechanism includes a sensor coupled to the mask body to detect pressure applied to the outer surface of the mask body and activate the blower assembly to deliver the breath to the subject.

In one example of the ventilation mask, the blower assembly is attachable to the outer surface of the mask body defining a portable ventilation system.

In one example of the ventilation mask, the blower assembly is in direct fluid communication with the inlet opening of the mask body.

In one example of the ventilation mask, the ventilation mask further comprises a detachable tube assembly extending between the inlet opening of the mask body and the blower assembly.

In one example of the ventilation mask, the mask body defines an air filter assembly that is in fluid communication with the inlet opening of the mask body.

In one example of the ventilation mask, the ventilation mask further comprises a jaw support assembly coupled to the mask body, wherein the jaw support assembly maintains a jaw posture of the subject during engagement of the inner surface of the mask body to the face of the subject.

In one example of the ventilation mask, the ventilation mask further comprises a capnography port in a wall of the mask body.

In one example of the ventilation mask, the ventilation mask further comprises an endoscope port in a wall of the mask body.

There has thus been outlined, rather broadly, the more important features of the technology so that the detailed description thereof that follows may be better understood, and so that the present contribution to the art may be better appreciated. Other features of the present technology will become clearer from the following detailed description of the technology, taken with the accompanying drawings and claims, or may be learned by the practice of the technology.

### BRIEF DESCRIPTION OF THE DRAWINGS

FIG. 1A is a drawing illustrating an exemplary portable ventilation device as disclosed herein.

FIG. 1B is a side view of the portable ventilation device of FIG. 1A.

FIG. 1C is a top view of the portable ventilation device of FIG. 1A.

FIG. 1D is a bottom view of the portable ventilation device of FIG. 1A.

FIG. 2 is a drawing that illustrates an example of one-handed operation of a ventilation device by a user as disclosed herein.

FIG. 3 is a drawing illustrating an exemplary portable ventilation device which can be secured to a wrist mount as disclosed herein.

FIG. 4A is a drawing that illustrates an exemplary portable ventilation device with a handle as disclosed herein.

FIG. 4B is a side front view of the portable ventilation device of FIG. 4A.

FIG. 4C is a side view of the portable ventilation device of FIG. 4A.

FIG. 4D is a top view of the portable ventilation device of FIG. 4A.

FIG. 5 is a drawing illustrating an example of attaching a ventilation device of FIG. 4 to an attachment point using an attachment mechanism coupled to the handle of the ventilation device as disclosed herein.

FIG. 6 is a drawing that illustrates exemplary aspects of a ventilation mask as disclosed herein.

FIG. 7 is block diagram illustrating example computing components of a ventilation device as disclosed herein.

These drawings are provided to illustrate various aspects of the technology and are not intended to be limiting of the scope in terms of dimensions, materials, configurations, arrangements or proportions unless otherwise limited by the claims.

### DETAILED DESCRIPTION

The present technology now will be described more fully hereinafter with reference to the accompanying drawings, in which some, but not all embodiments of the technology are shown. Indeed, the technology may be embodied in many different forms and should not be construed as limited to the embodiments set forth herein; rather, these embodiments are provided so that this disclosure will satisfy applicable legal requirements. Like numbers refer to like elements throughout. It is to be understood that this technology is not limited to the particular methodology and protocols described, as such may vary. It is also to be understood that the terminology used herein is for the purpose of describing particular embodiments only, and is not intended to limit the scope of the present technology.

Many modifications and other embodiments of the technology set forth herein will come to mind to one skilled in the art to which the technology pertains having the benefit of the teachings presented in the foregoing description and the associated drawings. Therefore, it is to be understood that the technology is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a”, “an”, and “the” include plural referents unless the context clearly dictates otherwise. For example, use of the term “a leak opening” can refer to one or more of such leak openings unless the context indicates otherwise.

As used herein, the terms “about” and “approximately” are used to provide flexibility, such as to indicate, for example, that a given value in a numerical range endpoint may be “a little above” or “a little below” the endpoint. The degree of flexibility for a particular variable can be readily determined by one skilled in the art based on the context. However, unless otherwise enunciated, the term “about” generally connotes flexibility of less than 2%, and most often less than 1%, and in some cases less than 0.01%.

As used herein, the terms “optional” or “optionally” mean that the subsequently described event or circumstance may or may not occur, and that the description includes instances where said event or circumstance occurs and instances where it does not.

The word “or” as used herein means any one member of a particular list and also includes any combination of members of that list.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Technologies are described for portable ventilation devices and systems. In one example configuration, a portable ventilation system comprises a ventilation mask that has a mask body with an outer surface and an inner surface configured for engagement with a face of a subject, and defines an inlet opening configured to receive an airflow. The portable ventilation system further comprises a blower assembly positioned in fluid communication with the inlet opening of the mask body, and the blower assembly is configured to direct an airflow to the inlet opening of the mask body. Further, the portable ventilation system includes a breath delivery mechanism, that when manually operated, activates the blower assembly and delivers a breath to the subject by increasing a pressure within the ventilation mask. The breath delivery mechanism allows for one-handed operation of the portable ventilation system, such that a user can selectively engage the breath delivery mechanism to initiate delivery of ventilation support to a subject. In one example, the breath delivery mechanism can comprise a sensor coupled to the mask body, and the sensor can be configured to detect pressure applied to the outer surface of the mask body and activate the blower assembly to deliver a breath to a subject. In another example, the breath delivery mechanism can comprise a button coupled to the mask body, and the button, when engaged, can be configured to operate the blower assembly to deliver a breath to a subject. A plurality of buttons can be provided on the mask. The buttons may be placed on both sides of the mask so that the operator can operate the mask with either right or left hand. In some cases, simultaneous actuation of both buttons can trigger operation of the mask in order to prevent accidental activation.

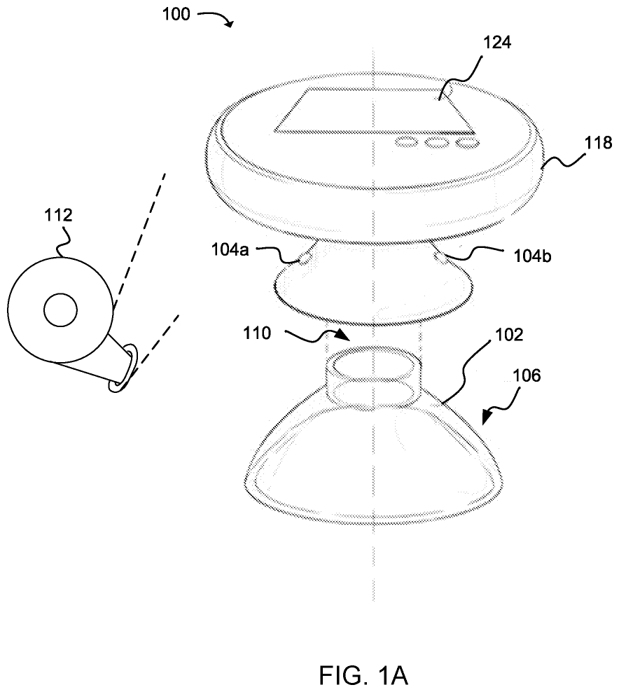

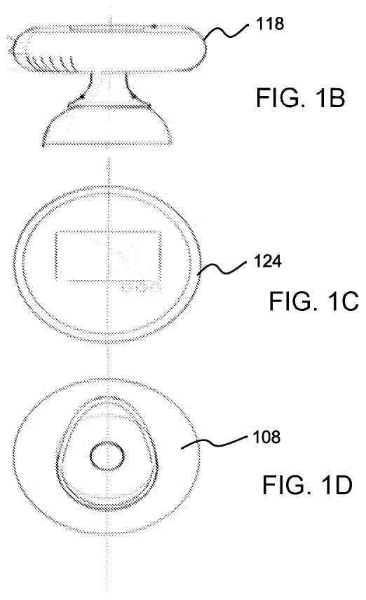

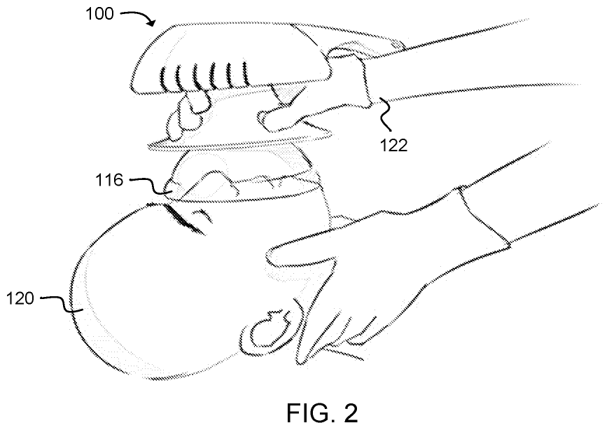

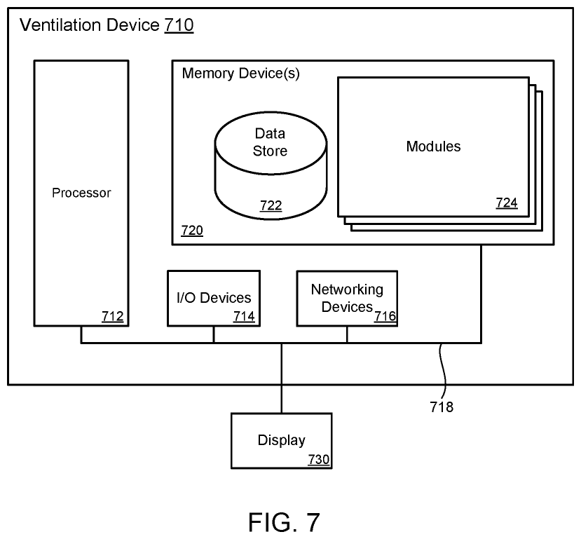

To further describe the present technology, examples are now provided with reference to the figures. FIGS. 1A-2 illustrate example portable ventilation devices 100. A portable ventilation device 100 can comprise a base unit 118 which houses a blower assembly 112 and a breath delivery mechanism 104a-b, a mask body 102, and other components operatively associated with the ventilation device 100. In one aspect, the blower assembly 112 can include a miniature or micro radial blower that generates an airflow. The blower assembly 112 can be positioned in the base unit 118 to direct the airflow to an inlet opening 110 defined by the mask body 102. For example, the base unit 118 can define a pathway to direct the airflow generated by the blower assembly 112 to the inlet opening 110 of the mask body 102. Optionally, it is contemplated that the blowing assembly 112 can deliver air to the inlet opening 110 of the mask 112 at a pressure of up to about 25 cm H₂O. The base unit 118 can also include a processor (shown in FIG. 7) positioned in operative communication with (e.g., communicatively coupled to, by either wired or wireless connection) the blower assembly 112. In operation, the processor can be configured to selectively control the blower assembly 112 based upon at least a measured pressure within the mask body 102, as described in patent publication US 2019/0070374 A1, published Mar. 7, 2019, which is incorporated herein by reference in its entirety.

The breath delivery mechanism 104a-b can be manually operated to activate the blower assembly 112 and deliver a breath to the subject 120 by increasing a pressure within the mask body 102. Delivering a breath to a subject 120 may refer to activating a blower in the blower assembly 112 to increase mask pressure and force air into the lungs of the subject 120 to deliver a tidal volume (i.e., the lung volume representing the normal volume of air displaced between normal inhalation and exhalation when extra effort is not applied) to the subject 120. In one aspect, the breath delivery mechanism 104a-b can include a sensor, button, or other input element located on the base unit 118 of the ventilation device 100, as illustrated in FIG. 1A. The breath delivery mechanism 104a-b can be positioned on the base unit 118 to allow one-handed operation of the ventilation device 100 by a user 122 (e.g., an emergency medical technician (EMT) or respiratory therapist), as illustrated in FIG. 2. The breath delivery mechanism 104a-b, in some aspects, can include a plurality of sensors, buttons, or other input elements positioned on the base unit 118, such that at least one breath delivery mechanism 104a-b may be within one-handed reach of a user 122. In another aspect, the breath delivery mechanism 104a-b can include one or more sensors directly coupled or integrated into the mask body 102. The sensors can detect squeezing pressure applied to the outer surface 106 of the mask body 102 (e.g., pressure applied to opposite sides of the outer surface 106 of the mask body 102) which activates the blower assembly 112 to deliver a breath to the subject 120. As an illustration, using one hand, a user 122 can hold the mask body 102 and position the ventilation device 100 to cover the mouth and nose of the subject 120, and using the one hand, the user 122 can squeeze the mask body 102 to deliver a breath to the subject 120, wherein the one or more sensors detect the squeezing pressure applied by the user 122 to the mask body 102 and activate the blower assembly 112 to deliver the breath to the subject 120. Notably, breath delivery can be accomplished without the use of an intervening tube or conduit which is typically present in ventilation devices. In particular, air is delivered directly from the blower assembly 112 to the inlet opening 110 of the mask body 102. Optionally, a secondary oxygen inlet can be oriented to allow supplemental oxygen to be introduced into the blower assembly or directly into the mask body. In this case, a removable supply tube can be connected to the secondary oxygen inlet. Such supplemental oxygen can be used during clinical procedures or after stabilization during a remote emergency.

The mask body 102 has an outer surface 106 and an inner surface 108 (shown in FIG. 1D) which is configured for engagement with a face of a subject 120. The mask body 102 defines an inlet opening 110 configured to receive an airflow generated by the blower assembly 112. Optionally, the mask body 102 can include a mask cushion 116 which can extend along and underneath at least a portion of the periphery of the mask body 102 such that the mask cushion 116 can be configured for engagement with the face of the subject 120 to provide a comfortable fit over the mouth and nose of the subject 120. It is contemplated that the mask body 102 can comprise any materials conventionally used for ventilation masks as are known in the art. Thus, the specific materials of the mask body 120 are not disclosed in detail herein.

In one aspect, the ventilation device 100 can include a jaw support assembly (not shown) coupled to the ventilation device 100 (e.g., to the base unit 118 and/or to the mask body 102). The jaw support assembly can maintain a jaw posture of a subject 120 during engagement of the mask body 102 to the face of the subject 120. In one aspect, the jaw support assembly can be placed under a subject's chin to maintain jaw posture during engagement of the inner surface 108 of the mask body 102 to the face of the subject 120. The jaw support assembly can be one or more of an elastic strap, a support tab that extends from a lower portion of the mask body 102, and the like. For example, a strap can extend around a head of the patient.

In further exemplary aspects, the ventilation device 100 can include a display device 124 configured to display information regarding one or more conditions of the ventilation system or a subject 120. Optionally, as shown in FIG. 1C, the display device 124 can comprise a user interface, such as, for example and without limitation, a touch screen display, keypads, keyboard, or voice recognition, as are known in the art. The display device 124 can be operatively associated with one or more components of the portable ventilation device 100. Further, the portable ventilation system 110 can include one or more hardware input controls which can be operatively associated with one or more components of the portable ventilation device 100.

The ventilation device 100 can include a battery assembly (not shown) to power the blower assembly 112 (as well as other ventilation system components) in response to the breath delivery mechanism 104a-b being manually operated. In one aspect, the battery assembly can be one or more rechargeable batteries which can provide power to the ventilation device 100, as an example, for at least eight hours on a single charge. Further, in one aspect, the battery assembly can be configured for wireless charging by including an induction coil and rectifier in the ventilation device 100 for inductive charging of the battery assembly.

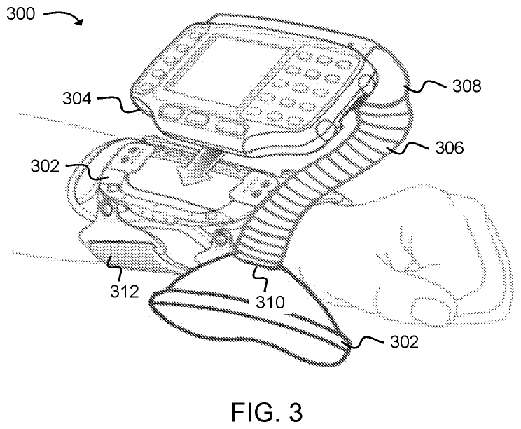

In further exemplary aspects, and as shown in FIG. 3, it is contemplated that a portable ventilation system 300 can be mounted to a wrist mount 302 to allow a user, such as an EMT or other rescue professional, to carry the ventilation system 300 on the user's wrist, making the ventilation system 300 readily accessible to the user when responding to an emergency medical request. In one aspect, the ventilation system 300 can include a base unit 304 comprising a housing that attaches or docks to the wrist mount 302. The wrist mount 302 can include one or more attachment or retention mechanisms (e.g., clips, tabs, friction retention device, and the like) that, when engaged with the housing of the base unit 304, secure the base unit 304 to the wrist mount 302, and allow for detachment of the base unit 304 from the wrist mount 302. Also, as shown in FIG. 3, the wrist mount 302 can include a strap 312 which can be wrapped or looped around a user's arm to secure the wrist mount 302 to the user's arm. The strap 312 can comprise an elongated strip or ribbon of textile, leather, or other flexible material having an attachment mechanism (e.g., a clip, clasp, buckle, or the like) used to fasten and secure the wrist mount 302 to the user's arm. Optionally, as can be appreciated, the strap 312 can be used to secure the wrist mount 302 to a user's body in other ways, such as wrapping the user strap around the user's waist or leg.

The base unit 304 can house the blower assembly (e.g., a miniature or micro radial blower) as described earlier, and the blower assembly can be positioned in the base unit 304 to direct an airflow generated by the blower assembly to an inlet opening 310 of a ventilation mask 302 to provide continuous positive airway pressure (CPAP). As shown in FIG. 3, in one aspect, the ventilation system 300 can include a tube assembly 306 that is attached at one end to the base unit 304 (i.e., an outlet opening 308 of the blowing assembly) and attached at the other end to the inlet opening 310 of the ventilation mask 302, such that the tube assembly 306 flexibly extends between the base unit 304 and the inlet opening 310 of the ventilation mask 302. The tube assembly 306 allows a user to hold ventilation mask 302 in one hand while the base unit 304 is attached to the wrist/arm of that hand and extend the tube assembly 306 so that the ventilation mask 302 can be placed over a subject's nose and mouth. It is contemplated that the tube assembly 306, in one aspect, can be detached from the outlet opening 308 of the blower assembly or the inlet opening 310 of the ventilation mask 302 to allow an alternative ventilation technique to be used (e.g., attach an intensive care unit ventilator, oxygen source, etc.). In another aspect, the ventilation mask 302 can be directly coupled to the base unit 304, such that an outlet opening 308 of the blowing assembly may be in direct fluid communication with the inlet opening 310 of the ventilation mask 302. In one aspect, the ventilation mask 302 may be removably attached to the base unit 304, such that a user can attach the ventilation mask 302 to the base unit 304 prior to treating a subject, and the user can detach the ventilation mask 302 from the base unit 304 when the ventilation system 300 is not in use.

Further, the base unit 304 can house a breath delivery mechanism which can be manually operated to activate the blower assembly housed in the base unit 304 and deliver a breath to a subject by increasing a pressure within the ventilation mask 302. In one aspect, the breath delivery mechanism can be positioned on the base unit 304 to allow a user to hold the base unit 304 in one hand, hold the ventilation mask 302 in the other hand, and operate the ventilation system 300 via the breath delivery mechanism to deliver a breath to the subject. The breath delivery mechanism can comprise a button or other input element positioned on the base unit 304. In another aspect, the breath delivery mechanism can include one or more sensors directly coupled or integrated into the ventilation mask 302. As described earlier, the sensors can detect squeezing pressure applied to an outer surface of the ventilation mask 302 and activate the blower assembly in the base unit 304 to deliver a breath to a subject.

The base unit 304 can be small and light, allowing the base unit 304 to be attached to the wrist mount 302. The base unit 304 can have low power requirements to allow the base unit 304 to be battery powered. The base unit 304 can include electronics to provide monitoring of a subject, such as respiratory rate, tidal volume, and delivered oxygen, as well as provide alarms to alert a user to any respiratory problems that the subject may experience. The ventilation mask 302 can be configured to fit nearly any subject, such that a tight, leak-proof seal may not be needed to increase pressure within the ventilation mask 302 and deliver a breath to the subject. As will be appreciated, it is contemplated that the base unit 304 can house other components which are operatively associated with the ventilation system 300.

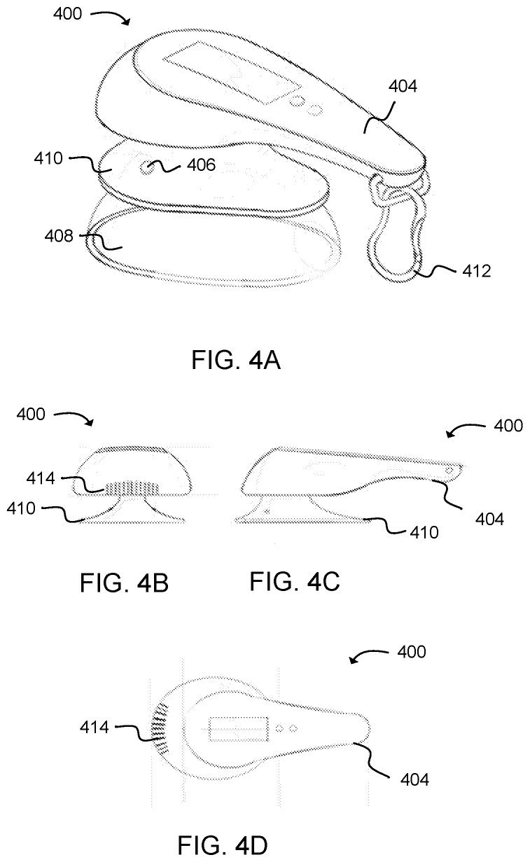

FIG. 4A-D illustrates another exemplary aspect of a portable ventilation system comprising a ventilation device 400 with a handle 404 having a frustoconical shape which extends away from a main body of the ventilation device 400. The ventilation device 400 can house a blower assembly (shown in FIG. 1A) positioned in the ventilation device 400 to be in fluid communication with an inlet opening of a ventilation mask 408 coupled to the ventilation device 400.

As shown in FIG. 4A, the ventilation device 400 can include a conical extension 410 which is shaped to direct an airflow generated by the blower assembly to the ventilation mask 408. In the aspect shown in FIG. 4A, a breath delivery mechanism 406 (e.g., a button) can be positioned on the conical extension 410, and the breath delivery mechanism 406 can be manually operated to activate the blower assembly to increase an air pressure within the ventilation mask 408 and deliver a breath to a subject, as described earlier. The breath delivery mechanism 406 can be positioned in multiple locations on the conical extension 410 to provide a user with increased access to the breath delivery mechanism 406. The conical extension 410 can be of a size and shape that allows a user to grasp the ventilation device 400 and manually operate the breath delivery mechanism 406 using one hand (e.g., by pushing a button) as illustrated in FIG. 2, allowing the user, for example, to use the other hand to position a subject's head in a position that opens up the subject's airway. Alternatively, in one aspect, it is contemplated that a breath delivery mechanism 406 (e.g., a button) can be positioned on the handle 404 of the ventilation device 400, allowing a user to hold the ventilation device 400 by the handle 404 and position the ventilation mask 408 to cover a subject's mouth and nose while simultaneously operating the breath delivery mechanism 406 to deliver breaths to the subject.

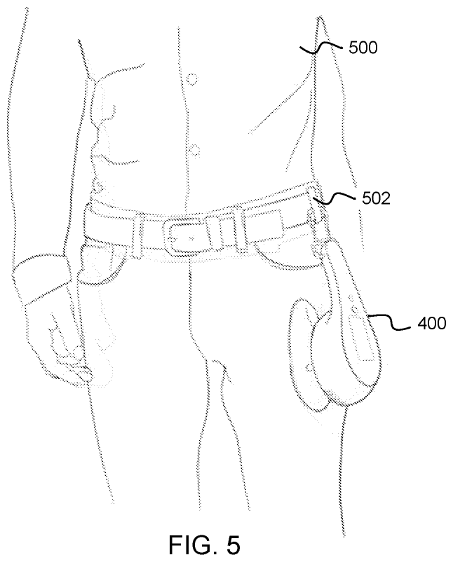

Further, as shown in FIG. 4B as one example, it is contemplated that the ventilation device 400 can include an air vent 414 to allow the blower assembly to draw ambient air through the air vent and direct the air to the ventilation mask 408. Also, in one exemplary aspect, the handle of the base unit 402 can include an attachment mechanism 412 (e.g., a clip, hook, snap, and the like) that allows the ventilation device 400, as shown in FIG. 5, to be attached to user 500 via, for example, an attachment point 502 (loop, hook, snap, and the like) located on a belt, vest, pants, shirt, backpack, medical bag, etc. Accordingly, the ventilation system can be easily carried in the field such that the ventilation system may be available to a user (e.g., EMT or other emergency responder) in an emergency situation.

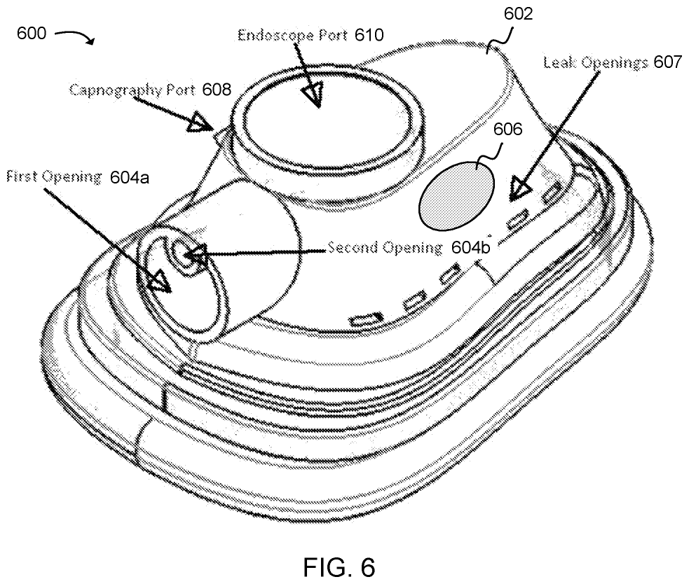

FIG. 6 illustrates exemplary aspects of a ventilation mask 600. The ventilation mask 600 includes a mask body 602 having an outer surface and an inner surface configured for engagement with a face of a subject. The mask body 602 defines an inlet opening 604a-b configured to receive air from a blower assembly, increasing air pressure within the mask body and delivering a breath to the subject. In one exemplary aspect, the ventilation mask 600 can coupled to the base unit shown in FIGS. 1-4 using a dual-lumen tube. One lumen can be used to deliver pressurized air from the base unit to the ventilation mask 600 and the other lumen can be used to monitor the air pressure inside the ventilation mask 600 by electronics included in the base unit.

In one exemplary aspect, the ventilation mask 600 can include a breath delivery mechanism 606, that when manually operated, activates a blower assembly (shown in FIG. 1A) to deliver a breath to the subject, as described earlier. The breath delivery mechanism 606 can be communicatively coupled to the blower assembly contained in a base unit via a wired or wireless connection, such that manually operating the breath delivery mechanism 606 located on the ventilation mask 600 sends an electrical signal to the base unit and activates the blower assembly. In one aspect, the breath delivery mechanism 606 can include a sensor coupled to the mask body 602 that detects pressure applied to the outer surface of the mask body 602 (e.g., squeezing pressure that slightly deforms the mask body 602) and activates the blower assembly to deliver a breath to the subject. In another aspect, the breath delivery mechanism 606 can include a button that is directly coupled to the mask body 602 (e.g., embedded into mask body 602) to operate the blower assembly to deliver the breath to a subject.

The ventilation mask 600, in one aspect, can include a jaw support assembly (not shown) coupled to the mask body 602, wherein the jaw support assembly can maintain a jaw posture of a subject during engagement of the ventilation mask 600 to the face of the subject. In one aspect, the jaw support assembly can be placed under a subject's chin to maintain jaw posture during engagement of the inner surface of the mask body 602 to the face of the subject. The jaw support assembly can be one or more of an elastic strap, a support tab that extends from a lower portion of the mask body 602, and the like. The ventilation mask 600 may also include attachment points (not shown) for one or more straps to hold the ventilation mask 600 on a subject's head.

The ventilation mask 600 can include one or more leak openings 607 that extend between the inner surface and the outer surface of the mask body 602 to control pressure within the ventilation mask 600 by allowing exhalation of CO₂ and smoother pressure signals while the blower maintains positive airflow. The leak openings 607 can be used to allow exhaled air to escape from the ventilation mask 600. The number, size, shape, and position of the leak openings 607 may vary, and may be configured to prevent blocking of the leak openings 607 by a user's hand. For example, in some cases the leak openings can be distributed along a periphery of the mask body such as at least over 50% of the periphery. The size of the leak openings 607 can vary so long as the leak openings 607 are large enough to allow for expiration without the need for excessive exerted pressure and small enough to allow buildup of sufficient pressure during inspiration and constant flow (CPAP). For example, the leak openings 607 can be a size in at least one dimension (e.g., a single dimension or a plurality of dimensions) ranging from about 1 to about 3 mm. It is contemplated that the leak openings 607 can be spaced about peripheral portions of the ventilation mask 600. Optionally, in these aspects, the leak openings 607 can be spaced evenly or substantially evenly about at least a portion of the periphery of the mask body 602. In exemplary aspects, it is contemplated that the leak openings 607 can be spaced radially inwardly from a peripheral edge of the mask body 602 by a distance ranging from about 1 mm to about 5 mm. In further exemplary aspects, it is contemplated that the spacing of the leak openings 607 can be determined based on the size of the mask body 602 (e.g., the circumference of the mask body 602) and the number of leak openings 607. In further exemplary aspects, it is contemplated that the leak openings 607 can be positioned such that the leak openings 607 cannot be fully blocked when the ventilation mask 600 is held by a user. In even further exemplary aspects, it is contemplated that the leak openings 607 can be positioned such that air flow into a subject's eyes is minimized or eliminated. For example, it is contemplated that the leak openings 607 can be evenly spaced from one another about the periphery of the mask body 602, with the exception of the two areas below the respective eyes of a subject. While traditional ICU ventilator systems use a tight fitting mask with a complex strap arrangement to minimize leaks and keep the mask in place, the ventilation mask 600 allows for air leaks, and a ventilation system that includes the ventilation mask 600 can adapt to large and varying leaks by dynamically tracking a leaked air volume and calculating a leak factor so that the leaked air volume can be predicted based on an air pressure within the ventilation mask 600. For example, a blower speed of a blower assembly can be dynamically adjusted to maintain a constant air pressure within the ventilation mask 600 under varying leak conditions. Because the ventilation mask 600 does not need a tight seal with the subject's face, the straps holding the ventilation mask 600 in place can be simpler than those used by ICU non-invasive ventilation systems.

In one aspect, the ventilation mask 600 can include a capnography port 608 in a wall of the mask body 602. The capnography port 608 may be provided in the ventilation mask 600 to allow a capnography sensor to measure CO₂ concentrations. The ventilation mask 600 may be provided with a removable cap for the capnography port 608 or the ventilation mask 600 may be molded with a tear-away cap that seals the capnography port 608 until the cap is removed.

In another aspect, the ventilation mask 600 can include an endoscope port 610 in a wall of the mask body 602. The endoscope port 610 allows an endoscope to enter the upper gastro-intestinal track of a subject through the mouth of the subject. The endoscope port 610 can be covered until it is needed. In one example, the endoscope port 610 may have a removable cap and may be sized to substantively conform to an endoscope to prevent excessive leakage of air around the endoscope. In another example, the endoscope port 610 may be covered with a penetrable material that creates an opening of adequate size as the endoscope is inserted through the endoscope port 610. The penetrable material may be a thin plastic material or other type of material that can be pierced by the endoscope itself or have an initial opening created by cutting a slit in the material and then inserting the endoscope. The covering material may conform to the endoscope to minimize the escape of air. Alternatively, the endoscope port 610 may have an opening sized for the endoscope with a self-sealing structure that allows the endoscope to enter, but adequately seals the opening if no endoscope is present. Optionally, the ventilation mask 600 may include a bite block integrated with the ventilation mask 600 to keep a subject's mouth open and allow the endoscope to enter the upper gastro-intestinal tract through the mouth.

In another exemplary aspect, the mask body 602 can define an air filter assembly (not shown) that is in fluid communication with the inlet opening 604a-b of the mask body 602. An air filter can be inserted into the filter assembly to filter out particulates that may be present in ambient air received from a blower assembly. In one example, the filter assembly can also include a HEPA filter.

The ventilation mask 600 can be used for procedures during which a subject's face can be obscured by the ventilation mask 600, such as a colonoscopy, a cardiac catheterization, minor podiatric surgery, and other like procedures. The ventilation mask 600 can be sized and shaped to cover the nose and mouth of most adults. However, because the ventilation mask 600 is configured to compensate for air leaks, the ventilation mask 600 need not conform closely to any particular subject's face. As such, the ventilation mask 600 can be used on nearly any adult without sizing or adjusting. Many patients undergoing conscious sedation with spontaneous respiration experience respiratory difficulties. Studies show that as many as half of these patients spend a portion of a procedure at an unsafe level of ventilation. Because of a higher rate of complications, high-risk patients are referred away from outpatient surgery centers to fully equipped hospitals where they can be monitored and provided with respiratory support through the use of an intensive care unit (ICU)-grade ventilator and staff. However, the added cost of such support may not be warranted for low and medium risk patients. The ventilation mask 600 can be used in out-patient surgery centers for low and medium risk patients, potentially saving costs associated with hospitals.

The ventilation devices and systems disclosed above can provide continuous positive airway pressure (CPAP), which can help prevent the airway from obstructing due to soft tissue collapse. In one aspect, the ventilation devices can monitor tidal volume, respiratory rate, and airway resistance. If a respiratory complication is detected, the ventilation devices can increase respiratory support to provide a breath to the patient and warn a clinician. In effect, the ventilation devices can reduce respiratory complications and replace the need for manually bag-ventilating a patient. The ventilation devices can be programmed to provide continual respiratory support and monitor tidal volume, respiratory rate, and airway resistance even in the presence of large or varying leaks around the ventilation mask. Using a digitally controlled miniature radial blower and a strategy to minimize power consumption, the ventilation devices can be small (e.g., 8×6×2 inches), can operate without compressed gas, and can run on batteries for an extended period of time (e.g., up to 8 hours).

The ventilation devices can use a change in mask pressure to force gas into the lungs and deliver the tidal volume. It is contemplated that the ventilation devices can inform a clinician of whether an exhaled breath volume, in response to a pressure change, is sufficient to maintain adequate ventilation. It is further contemplated that the ventilation devices can measure the flow of supplemental oxygen and can calculate the resulting inspired oxygen fraction (FiO2) delivered to a patient.

Accordingly, the ventilation devices of the present disclosure can use a high performance miniature radial blower to generate precise flows and pressures under microprocessor control. A pressure sensor (not shown) can measure pressure in a ventilation mask and system software can control the speed of the blower to provide precise air pressure in the ventilation mask regardless of mask leak. The ventilation devices can ventilate a subject by periodically raising the air pressure so that gas can be forced into the subject's lungs. The volume of each breath can be determined by the amount of pressure support, and the subject's lung (and chest wall) compliance. In a typical patient with compliance of 50 ml/cm H2O, pressure support of 10 cm H2O may result in a 500 ml breath. It is contemplated that the ventilation devices can deliver pressure support breaths of up to 25 cm H2O.

The ventilation devices can include one or more flow sensors (not shown) positioned in communication with a processor (not shown) configured to measure an air flow rate at which air is provided from a blowing assembly to an inlet opening of the ventilation mask 600. A flow sensor can be positioned in fluid communication with the blowing assembly and the ventilation mask 600. The processor can be configured to calculate one or more of:
A leak flow rate (e.g., Leak Flow Rate=the flow rate at which air exits leak openings 607 of the mask body 602; and Mask Pressure=the measured pressure within the mask body 602); and
A patient flow rate (e.g., Patient Flow Rate=the flow rate of gas inhaled by a subject; and Total Flow Rate=the flow rate of gas supplied to the inlet opening 604a-b of the mask body 602).

In one aspect, the processor can be configured to determine a respiratory rate of a subject based upon measured changes in the pressure within the mask body 602. In these aspects, it is contemplated that the processor can be configured to determine a tidal volume of each breath of the subject based upon the determined patient flow rate. In still further exemplary aspects, the processor can be configured to determine a leak flow rate for each respective breath of the subject.

As an example, a processor with non-volatile memory as well as working memory can be included on a printed circuit board in the base unit. The processor can include analog-to-digital (ADC) inputs which are coupled to the multiple pressure sensors as well as a battery level to allow the processor to obtain readings from those devices in real-time. The processor can also have outputs to control driver electronics for the blower which allows the processor to control the speed of the blower. An output driven by a digital-to-analog converter (DAC) may be used to provide an analog signal controlling the speed of the blower in some cases.

Software loaded into the non-volatile memory and running on the processor enables many different functions in the base unit. Several functions depend on understanding when the patient is inhaling and exhaling. Therefore, a breath detection algorithm can determine these periods by finding several key markers in the signal from the blower airflow sensor. For example, a first breath mark (BM1) that corresponds to the start of inspiration and a new breath can be determined. The first breath mark can correspond to a point when the flow signal passes above a flow baseline plus a threshold value that can be adjusted according to desired settings. The software also determines a second breath mark (BM2) that corresponds to the end of inspiration and the start of expiration. The second breath mark can correspond to a point when the flow signal passes through the flow baseline. The software then determines a third breath mark (BM3) that corresponds to the end of the breath. This third breath mark can correspond to a point when the flow signal passes through the flow baseline less the threshold value. The time from BM3 to the next BM1 is the time between breaths. The software can determine the overall respiration rate by calculating the time between BM1 marks.

In this example, the software also dynamically calculates a leak factor for the amount of air currently leaking from the mask as a function of the mask pressure. In the time between breaths, all of the flow from the blower is escaping through leaks in and around the mask. The output of the blower airflow sensor (plus the oxygen flow sensor if used) is averaged over the time period from BM3 and BM1 to determine a current flow baseline. A dynamic leak factor can be recalculated for each breath by dividing the average leak flow by the mask pressure.

The tidal volume for the patient can also be calculated by the software. A patient flow can be calculated throughout the breath as the total flow from the blower (plus oxygen flow) minus the leak flow which can be dynamically calculated as the leak factor times pressure. The patient flow is then integrated by the software over the inhalation portion of the breath (BM1 to BM2) to determine the tidal flow for that breath.

Similarly, the inspired oxygen fraction (FiO₂) can be calculated if an oxygen source is coupled to the unit. The blower airflow rate is assumed to have an oxygen content of 21% so that can be combined with the oxygen flow rate at 100% oxygen to calculate the oxygen saturation (FiO₂) of the air being provided to the patient.

In one example, the software can control the blower using a proportional-integral-derivative (PID) technique. The software can use the mask pressure in a feedback loop to control the fan speed to maintain the mask pressure at a desired level, which can be adjusted by the clinician. The control loop is tuned to create a responsive system that can adapt quickly to changing leaks inside the mask while also not overshooting pressures when ramping up that could over pressurize the patient's airway and cause injury.

In normal CPAP mode, a single pressure level can be maintained, but in a BiPAP mode, two different pressure levels are created with the higher pressure level used to augment a breath by the patient. Optionally, an automatic BiPAP mode can be provided where a higher pressure pulse is initiated at a regular interval (adjustable by the clinician), to stimulate breathing in a patient that is not spontaneously breathing. In another alternative, the control system can automatically switch from a CPAP mode to an automatic BiPAP mode if no breathing by the patient is detected for a certain clinician-adjustable time period.

The software can also use the mask pressure and flow rates to determine whether or not the mask is placed on the patient's face and use this information to automatically start and/or stop the blower. If the mask pressure is the same as atmospheric pressure with little to no variation over a multi-breath period of time, it can be assumed that the mask is not on the patient's face and the blower can be turned off to reduce noise and power. In addition, or alternatively, an alarm can be sounded to alert the clinician that the mask is no longer on the patient's face. If the blower is off and a change in mask pressure is detected caused by the patient breathing, the blower can be automatically turned on to start providing CPAP.

FIG. 7 illustrates computing components which can be included in a ventilation device 710. The ventilation device 710 can include one or more processor 712 that are in communication with memory devices 720. The ventilation device 710 can include a local communication interface 718 for the components in the ventilation device 710. For example, the local communication interface 718 can be a local data bus and/or any related address or control busses as may be desired.

The memory device 720 can contain modules 724 that are executable by the processor 712 and data for the modules 724. In one example, the memory device 720 can include an operating system module, a leak flow rate module, a patient flow rate module, and other modules. The modules 724 can execute the functions described earlier. A data store 722 can also be located in the memory device 720 for storing data related to the modules 724 and the operating system that is executable by the processor 712.

The ventilation device 710 can also have access to I/O (input/output) devices 714 that are usable by the computing components. An examples of an I/O device 714 include a display 730, a sensor, and/or button. In some aspects, the ventilation device 710 can include a networking device 716 and similar communication devices to allow the ventilation device 710 to communicate with a computer, server, cloud service, etc. over a computer network. The networking devices 716 can be wired or wireless networking devices that connect to the internet, a LAN, WAN, or other computing network.

The components or modules that are shown as being stored in the memory device 720 can be executed by the processor 712. The term “executable” can mean a program file that is in a form that can be executed by the processor 712. For example, a program in a higher level language can be compiled into machine code in a format that can be loaded into a random access portion of the memory device 720 and executed by the processor 712, or source code can be loaded by another executable program and interpreted to generate instructions in a random access portion of the memory device 720 to be executed by the processor 712. The executable program can be stored in any portion or component of the memory device 720. For example, the memory device 720 can be random access memory (RAM), read only memory (ROM), flash memory, a solid state drive, memory card, a hard drive, or any other memory components.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

What is claimed is:

1. A ventilation mask, comprising:
a mask body having an outer surface and an inner surface configured for engagement with a face of a subject, and an inlet opening to receive air from a blower assembly; and
a breath delivery mechanism, that when manually operated, is configured to activate the blower assembly to deliver a breath to the subject.

2. The ventilation mask in claim 1, wherein the breath delivery mechanism includes a sensor coupled to the mask body to detect pressure applied to the outer surface of the mask body and activate the blower assembly to deliver the breath to the subject.

3. The ventilation mask in claim 1, wherein the breath delivery mechanism includes a button directly coupled to the mask body to operate the blower assembly to deliver the breath to the subject.

4. The ventilation mask in claim 1, wherein the blower assembly is attachable to the outer surface of the mask body defining a portable ventilation system.

5. The ventilation mask in claim 4, wherein the blower assembly is in direct fluid communication with the inlet opening of the mask body.

6. The ventilation mask in claim 1, further comprising a detachable tube assembly having a first end configured to couple to the inlet opening of the mask body, and a second end configured to couple to the blower assembly.

7. The ventilation mask in claim 1, wherein the mask body defines an air filter assembly that is in fluid communication with the inlet opening of the mask body.

8. The ventilation mask in claim 1, further comprising a jaw support assembly coupled to the mask body, wherein the jaw support assembly maintains a jaw posture of the subject during engagement of the inner surface of the mask body to the face of the subject.

9. The ventilation mask in claim 1, further comprising a capnography port in a wall of the mask body.

10. The ventilation mask in claim 1, further comprising an endoscope port in a wall of the mask body.

11. A portable ventilation system, comprising:
a ventilation mask, wherein the ventilation mask includes a mask body having an outer surface and an inner surface configured for engagement with a face of a subject, wherein the mask body defines an inlet opening configured to receive an airflow;
a blower assembly positioned in fluid communication with the inlet opening of the mask body to direct the airflow to the inlet opening of the mask body; and
a breath delivery mechanism, that when manually operated, activates the blower assembly and delivers a breath to the subject by increasing a pressure within the ventilation mask.

12. The portable ventilation system in claim 11, wherein the breath delivery mechanism includes a sensor coupled to the ventilation mask, and the sensor detects squeezing pressure applied to opposite sides of the outer surface of the mask body and activates the blower assembly to deliver the breath to the subject.

13. The portable ventilation system in claim 11, wherein the breath delivery mechanism includes a button directly coupled to the ventilation mask, and the button activates the blower assembly to deliver the breath to the subject.

14. The portable ventilation system in claim 11, further comprising a housing configured to contain the blower assembly, wherein the housing has dimensions and an attachment that allows the housing to be attached to a fastening worn by a person.

15. The portable ventilation system in claim 11, further comprising:
a wrist mount configured to attach to a wrist and receive a housing containing the blower assembly for removable attachment to the wrist mount; and
a tube assembly having a first end configured to attach to the blower assembly and a second end configured to attach to the inlet opening of the mask body.

16. The portable ventilation system in claim 11, wherein the blower assembly is attached to the outer surface of the mask body and the blower assembly is in direct fluid communication with the inlet opening of the mask body.

17. The portable ventilation system in claim 11, further comprising a housing containing the blower assembly and the housing is configured to be removably attached to the outer surface of the mask body.

18. The portable ventilation system in claim 17, further comprising a tube assembly having a first end configured to attach to the inlet opening of the mask body and a second end configured to removably attach to the blower assembly contained in the housing or to an alternative ventilation mechanism.

19. The portable ventilation system in claim 11, further comprising a battery assembly to power the blower assembly in response to the breath delivery mechanism being operated.

20. The portable ventilation system in claim 19, wherein the battery assembly includes an induction coil and rectifier for inductive charging of the battery assembly.

21. The portable ventilation system in claim 11, further comprising a handle having a mask interface to connect with the inlet opening of the mask body, and shaped to allow a user to retain the ventilation mask on a patient using a single hand, while simultaneously engaging the breath delivery mechanism with the single hand.

22. The portable ventilation system of claim 21, wherein the handle has a frustoconical shape which extends away from a main body of the blower assembly.

23. The portable ventilation system in claim 11, further comprising a jaw support assembly configured to be placed under a subject's chin to maintain jaw posture during engagement of the inner surface of the mask body to the face of the subject.

25. The portable ventilation system in claim 11, wherein the mask body further comprises at least one leak opening extending between the inner surface and the outer surface of the mask body to control pressure within the ventilation mask.
